(19) United States
(12) Patent Application Publication
(10) Pub. No.: US 2025/0339102 A1
(43) Pub. Date: Nov. 6, 2025

(54) INNER SPEECH RECOGNITION METHODS AND APPARATUSES

(71) Applicants: Mokhles M. Abdulghani, Jackson, MS (US); Wilbur L. Walters Walters, Jackson, MS (US); Khalid H. Abed, Jackson, MS (US)

(72) Inventors: Mokhles M. Abdulghani, Jackson, MS (US); Wilbur L. Walters Walters, Jackson, MS (US); Khalid H. Abed, Jackson, MS (US)

(73) Assignee: Jackson State University, Jackson, MS (US)

(21) Appl. No.: 18/656,193

(22) Filed: May 6, 2024

(51) Int. Cl. A61B 5/00; A61B 5/28; A61B 5/377

(52) U.S. Cl. CPC A61B 5/7264; A61B 5/28; A61B 5/377

(57) ABSTRACT

The present disclosure relates to methods and apparatuses for classifying internalized speech using a heart-computer interface. In particular, disclosed herein is a method for interpreting electrocardiogram (ECR) signals in an individual using electrodes placed on the individual's skin. The method disclosed herein may be performed using a low-cost, low-channel ECG apparatus, such as by placing three sensors on the individual's skin and which may be wearable and portable to facilitate its use. The sensors may be placed on the left and right sides of the individual's forehead and on the left side below the individual's neck to collect ECG signals, although other placements are possible. features. Autoregressive coefficient (AR), Shannon entropy, fractal measures, and multiscale wavelet variance estimation may then be applied to the collected ECG signals to determine the individual's internalized speech.

### BACKGROUND OF THE INVENTION

There remains an ongoing communication problem for individuals with disabilities, such that these individuals often are unable to express themselves effectively. This includes individuals with conditions such as ALS, cerebral palsy, or speech disorders. Similarly, expression can be problematic even in unaffected individuals in multilingual settings, such that language barriers exist that prevent effective expression.

The term ‘affective’ is a psychological expression referring to the experience of human feelings and emotions. In 1995, the field of affective computing was first originated by Dr. Picard, who discussed neurological studies related to human emotions or other affective phenomena and the possibility of mimicking them with a computer using the concept of emotion recognition. The type of speech or words a person produces is essentially linked with the internal affections or emotional experience that person is going through. As a result, recent studies on determining intended expression have focused on processing physiological signals in a multimodal approach by combining different types of physiological signals such as electroencephalogram (EEG), electromyogram (EMG), galvanic skin response (GSR), blood volume pressure (BVP), photoplethysmography (PPG), or electrocardiogram (ECG).

Research has revealed that the heart is the most potent source of the electrical field in the human body. The amplitude of the electrical field generated by the heart can be 60 times higher than the electrical field generated by our brain. In addition, they stated that the nervous system acts as an antenna that responds and tunes to the magnetic fields generated by the heart. More research to enhance this energetic communication ability can result in a much deeper level of non-verbal communication between people, such as inner speech. The electrical field generated by the heart is monitored and measured through a process called electrocardiogra which records it in an ECG graph illustrating the variation in voltage versus time. ECG electrodes can be placed anywhere on the body's surface, capturing the dynamic response of the autonomic nervous system towards each emotion which is reflected as rhythmic fluctuation in the heart, and it can be recorded using a less mobile, intrusive, and wearable device. No study, however, has been published wherein there was an attempt to study or classify inner speech, imagined speech, or human thinking in general based on ECG alone in a unimodal approach (i.e., using a single type of signal).

Similarly, an enormous body of research has been conducted aiming to convert human brain signals to speech. Although experiments have shown that the excitation of the central motor cortex is elevated when visual and auditory cues are employed, the functional benefit of such a method is limited. Imagined speech, sometimes called inner speech, is an option for decoding human thinking using the brain-computer interface (BCI) concept. BCI is being developed to progressively allow paralyzed patients to interact directly with their environment. Brain signals usable with the BCI systems can be recorded with a variety of common recording technologies, such as magnetoencephalography (MEG), electrocorticography (ECOG), functional magnetic resonance imaging (fMRI), functional near-infrared spectroscopy (fNIRS), and electroencephalography (EEG). EEG headsets are used to record the electrical activities of the human brain. EEG-based BCI systems can convert the electrical activities of the human brain into commands.

Although some studies have focused on EEG alone, such studies have tended to suffer from poor accuracy and/or require the use of high-cost, high-channel headsets. No studies have attempted to study or classify inner speech, imagined speech, or human thinking in general based on ECG. As such, a low-cost, high-accuracy ECG solution would serve unmet needs in this field.

### SUMMARY OF THE INVENTION

In some embodiments, the present disclosure describes an internalized speech recognition method using a unimodal signal comprising the steps of: placing at least one electrode on an individual; collecting ECG data from the individual using the at least one electrode; extracting features from the collected ECG data using at least one feature extraction method; and classifying the features using supervised learning using a machine learning algorithm. In some embodiments, the features are classified using a predetermined set of words. For example, the predetermined set of words can be “yes” and “no” if a positive or negative response is desired. As yet another example, the predetermined set of words can be “north”, “south”, “east”, and “west” if a directional response is desired. As yet another example, the predetermined set of words can be “left”, “right”, “stop”, and “start” if movement commands are desired.

In some embodiments, the method further comprises the step of preprocessing the collected ECG data prior to extracting features, wherein the preprocessing comprises noise attenuation and calibration.

In some embodiments, the noise attenuation and calibration comprises the steps of applying a 4th order Butterworth bandpass filter with 0.5 Hz to 150 Hz bandwidth, applying a notch filter at 60 Hz, and applying a high-pass filter with a cut-off frequency of 0.5 Hz.

In some embodiments, the method further comprises presenting an audio or visual prompt to the individual prior to collecting ECG data. For example, an audio recording that poses a question to the individual may be played. Alternatively, text may be presented on a screen wherein the text asks the individual a question. As yet another alternative, an image or video may be displayed.

In some embodiments, the at least one electrode comprises three electrodes. In some embodiments, the electrodes are placed on the left side of the individual's forehead, the right side of the individual's forehead, and on the individual's left side anywhere below the neck.

In some embodiments, the at least one feature extraction method is selected from the group consisting of: autoregressive coefficient (AR), Shannon entropy, fractal measures, and multiscale wavelet variance estimation. In some embodiments, the at least one feature extraction method comprises autoregressive coefficient (AR), Shannon entropy, fractal measures, and multiscale wavelet variance estimation.

In some embodiments, the machine learning algorithm is support vector machine (SVM).

In some embodiments, the individual has been diagnosed with or suspected of having a speech disorder. In some embodiments, the speech disorder is mutism. In some embodiments, the individual has a disability that prevents or inhibits coherent speech. Alternatively, the individual may not speak a particular language.

### BRIEF DESCRIPTION OF THE DRAWINGS

The disclosure can be better understood with reference to the following drawings. The elements of the drawings are not necessarily to scale relative to each other, emphasis instead being placed upon clearly illustrating the principles of the disclosure. Furthermore, like reference numerals designate corresponding parts throughout the several views.

FIG. 1 depicts an overview of an embodiment of an HCI system for inner speech classification.

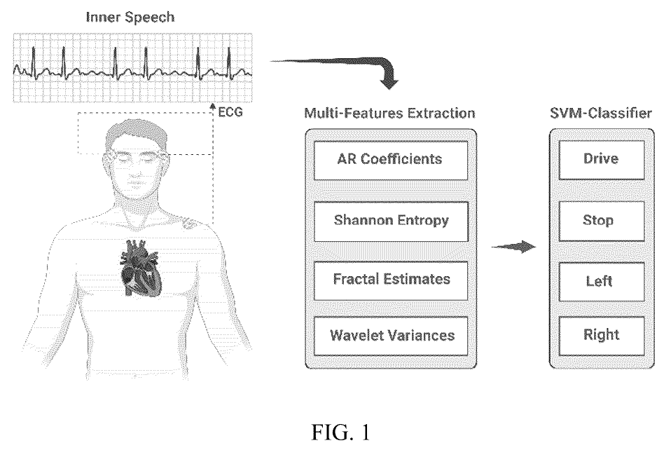

FIG. 2 depicts an embodiment of a method to collect ECG data.

FIG. 3 depicts a sample ECG recording with the obtained representation after using AR coefficients (feature 1-4), SE (feature 5-20), fractal estimates (feature 21-22), and MWV (feature 23-32).

FIG. 4 depicts a boxplot showing a sample of the variance between each class using the extracted features.

FIG. 5 depicts ROC-AUC of the SVM classifier using the extracted features.

FIG. 6 depicts ROC-AUC of the SVM classifier using DFT features.

### DETAILED DESCRIPTION

The present disclosure relates to methods and apparatuses for classifying internalized speech using a heart-computer interface. In particular, disclosed herein is a method for interpreting electrocardiogram (ECR) signals in an individual using electrodes placed on the individual's skin. The method disclosed herein may be performed using a low-cost, low-channel ECG apparatus, such as by placing three sensors on the individual's skin and which may be wearable and portable to facilitate its use. The sensors may be placed on the left and right sides of the individual's forehead and on the left side below the individual's neck to collect ECG signals, although other placements are possible. features. Autoregressive coefficient (AR), Shannon entropy, fractal measures, and multiscale wavelet variance estimation may then be applied to the collected ECG signals to determine the individual's internalized speech.

The examples, applications, descriptions and content disclosed herein are exemplary and explanatory, and are non-limiting and non-restrictive in any way.

All scientific terms used herein have the same meaning as commonly used and understood by one of ordinary skill in the art. Examples, materials, methods, figures and tables are illustrative only and not intended to be limiting.

As used herein, “AR” means autoregressive coefficient.

As used herein, “AUC” means area under curve.

As used herein, “DFT” means Discrete Fourier Transformation.

As used herein, “ECG” means electrocardiogram.

As used herein, “EEG” means electroencephalogram.

As used herein, “internalized speech” means an individual's thoughts or emotions that are not expressed audibly. For example, internalized speech can include, but is not limited to, an individual's thoughts.

As used herein, “ROC” means Receiver Operating Characteristic.

As used herein, “SVM” means support vector machine.

The present disclosure discloses classifying inner speech using ECG measurements of heart electrical activity. When using EEG for inner speech recognition, Applicants determined that applying excessive filtration on the raw EEG data damages features of the EEG signals and is associated with a significant drop in the classification accuracy. See Abdulghani, M. M.; Walters, W. L.; Abed, K. H. Imagined Speech Classification Using EEG and Deep Learning. Bioengineering 2023, 10, 649, which is incorporated herein by reference in its entirety. Typical EEG frequency bands and their approximate spectral boundaries are delta (1-3 Hz), theta (4-7 Hz), alpha (8-12 Hz), beta (13-30 Hz), and gamma (30-100 Hz). On the other hand, the frequency range for an ECG can be from 0.5 (or 0.05) to 100 (or 150) Hz. Since the entire ECG frequency range covers or overlaps all possible EEG frequency bands, it is possible that one may not distinguish whether the raw signal being recorded is an ECG or an EEG signal. For example, a 10-Hz alpha signal cannot be distinguished from a 10-Hz ECG signal since they have the same instantaneous frequency.

Therefore, the use of ECG is an extension of the crucial role of extracting features from an 8-channel EEG-based inner speech data with different feature extraction methods. Use of the EEG showed that the selected feature extraction techniques simplified the data and reduce the computation power needed in data training and validation. Moreover, there is a significant impact of simplifying the processed data on the possibility of operating the designed classifier in a realistic environment where it considerably impacts the product's producibility and cost when manufacturing them and converting them to a natural system. In the assessment of ECG, Applicants have added one more feature extracting technique to the same features extraction techniques applied in EEG, with different parameters and levels, and have applied them all to the recorded ECG data instead of EEG.

In this disclosure, Applicants used auditory stimuli during the recording of ECG for inner speech recognition. In addition, Applicants added visual stimulus by using a video game that helped the subjects to interact more efficiently with the experimental environment and generate classifiable ECG for the specified internally spoken commands. The subjects expressed their need for steering the car with internally spoken commands, which are either drive, stop, or turning right or turning left. Applicants gathered the recorded data from all the subjects and applied the disclosed SVM classification model to the recorded data. Although, the accuracy achieved in this work indicates that most of the inner speech commands were distinguished between Drive, Stop, Right, and Left, the command Stop was incorrectly classified as Drive or Right. The other commands states were also incorrectly predicted. This misclassification is mainly due to the composite and subjective nature of emotions. Some researchers suggested that finding common features among different subjects' heartbeats is difficult to be achieved. Moreover, the results showed that using a few seconds of ECG is sufficient to classify human thinking during inner speech activity rather than using one minute.

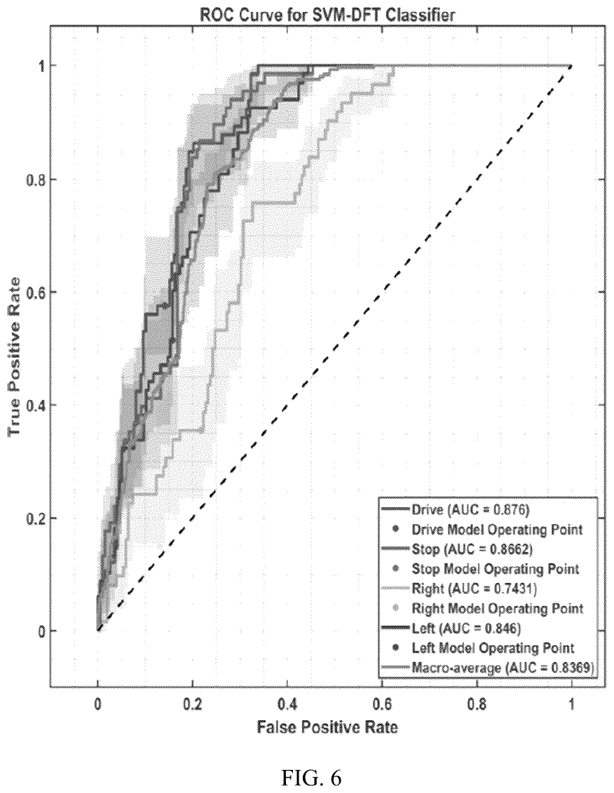

Applicants compared the feature-extraction method with the DFT feature-extraction method to show the advantages of the disclosed classification method. Applicants retrained and revalidated their model using a different feature extracting technique, and then Applicants applied the SVM to the ECG data, which was comprised of a 704-by-5000 matrix. Applicants obtained the magnitude DFT coefficients for each recording to perform the analysis in the frequency domain, and the performance of the resulting classifier was summarized as shown in Table 2 and FIG. 6.

TABLE 2
THE MODEL PERFORMANCE OF THE SVM
MODEL WITH THE DFT FEATURES

| | Precision | Recall | F1 Score |
|---|---|---|---|
| DRIVE | 57.576 | 57.576 | 57.576 |
| STOP | 48.529 | 50 | 49.254 |
| RIGHT | 35.484 | 33.333 | 34.375 |
| LEFT | 51.471 | 53.03 | 52.239 |

Although Applicants could achieve a noticeable data reduction using DFT and overall accuracy of 53.03%, that is still about 35% less than the overall accuracy obtained with the disclosed 32 features. These analyses show that the SVM classifier has benefited from the carefully selected features using Applicants' disclosed methods.

Besides the reduction in the size and complexity of data, the Applicants found a significant variance between the specified classes. Even though this is a significant reduction in data size and complexity, the main objective of using the disclosed multi-feature extraction method was not just a reduction in data. Applicants aimed to re-represent the data with a much smaller set of features that allows capturing the differences between the required classes, so a classifier could perfectly separate the ECG signals. As disclosed in the example section below, the extracted features resulted in high accuracy, precision, recall, F-score, and macroaverage AUC. The resulting classifier can be converted to a C++ or Python code using MATLAB code generation and uploaded to a microcontroller to be tested in real-time. The designed HCI system will enable a large segment of people, specifically paralyzed people with a speech disability, to interact with the outside world easily.

The disclosed methodologies offers many advantages over conventional methodologies including, but not limited to: lower cost equipment, less complex data sets to process, and superior accuracy for determining inner speech.

### Example—ECG Processing

### Materials and Methods

Subjects. Ten healthy and native English speaker subjects, aged from 22 to 55, participated as volunteers in the study. Each participant was asked to fill out a self-report questionnaire form illustrating their health history. The reported information in the questionnaire forms showed that none of them had speech or movement disorders, vision problems, a history of injury in the auditory canal or full, or partial hearing loss. Moreover, none of them experienced any cardiovascular, chronic, or mental disease. None of the participants had any previous contribution or experience with ECG recording, and they were classified by aliases “S1” through “S10”. Participants were informed about the purpose of the experiment and about the protocol to be followed in the recording sessions. During the experiment, participants were seated in a comfortable chair in a soundproofing environment while announcing the audio and video cues. In the experiment room, two of Applicants' research members stayed with each subject where the experiment took place for the whole duration of the ECG recording sessions. The study was conducted in the Department of Electrical & Computer Engineering and Computer Science at Jackson State University. Data were collected in accordance with approved Institutional Review Board (IRB) procedures at Jackson State University.

Apparatus. Three pre-gelled disposable electrodes type, Kendall H124SG Ag/AgCl, were used to acquire three ECG signals. These electrodes come with excellent adhesion to guarantee a good quality signal while being gentle on the skin. The flexible foam backing material for these sensors and the round shape ensure a good fit for most patients and ease the use and comfort during the signal acquisition process. The three sensors were connected to the same acquisition device using three clip-leads, 150 cm, 1.5 mm Snap-On connector. A wearable amplifier type g.Nautilus PRO FLEXIBLE, manufactured by g.tec, was used for acquiring the signals. This amplifier is a certified device by the Conformité Européenne (CE-certified), and the device was cleared by the United State Food and Drug Administration (FDA-cleared). The device is also capable of acquiring high-resolution physiological signals with 0.5 KHz and streaming them wirelessly to a nearby computer that can be used through the MATLAB software to visualize the acquisition in real-time. All g.tec amplifiers are designed to be connected to the input channels to enable synchronous and simultaneous recording of many electrophysiological data (including EEG, ECG, EMG, EOG, and ECOG). The computer used in this study has an AMD Ryzen 9—5950X/3.4 GHz processor, MSI Geforce RTX 3090-24 GB graphics card, CORSAIR Dominator Platinum 128 GB DDR4 memory, and Crucial P3—SSD (NVMe)—4 TB drive. A 55-inche high-resolution screen, in-ear headphones, and a car racing video game were used to generate the required auditory and video cues.

Experimental Setup. The inner speech was comprised of 1760 sessions in total for all the chosen commands. In each session, the subject was seated in the chair, putting on the in-ear headphones through which the auditory cue was announced. To familiarize the participant with the experimental procedures, all experiment actions were explained before the experiment date and before signing the consent form.

The experimental procedures were explained again during the experiment day while the ECG electrodes were placed. The setup of the electrodes and other devices took approximately 15 minutes. The participants were trained on the experiment procedure by conducting a demo session prior to the original one. Implementing a demo session was beneficial to get the subjects more adapted to the experimental procedure. In the demo session, Applicants focused on training each participant to avoid blinking, relax, take slow inhaling when starting to perform the inner speech, and try to breathe as slowly as possible until the end of the recording. Although the session time aimed for recording was 60 s, which is the recommended time by physiologists for eliciting emotion, the demo session showed that limiting the session time to 15 s can help obtain a better-quality signal with fewer motion artifacts. Each recording took 15 s, but the first 5 s of the recording were not included in the final dataset. The first 5 s were used to allow enough time for the subject to be emotionally engaged with the visual and audio cues. Subjects were seated in high-back chairs to lessen the postural effects on the positive ECG electrodes.

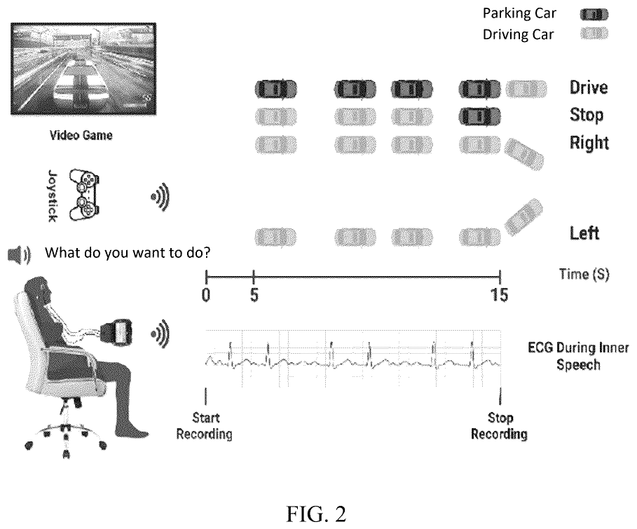

Opposite each subject, there was a screen displaying the racing car, where a racing car video game was used as a visual cue, and each subject was instructed to focus on the car during the speech imagery. In the beginning of the experiment, the video game was started, and the car was prepared to be either in stopping (parking) or moving (driving) condition. To implement the Drive command, the car was initiated to be in a stopping condition. For the other three commands, the car was initiated to be in the moving condition. When the recording started, the audio clip “What do you want to do?” was announced, and the participant started performing the specified command as inner speech. Each of those commands was repeated for 15 seconds, and the recording was stopped by the end of the 15 seconds session. Once the recording stopped and while the subject was still looking at the car, one of Applicants' research team members steered the car using a joystick according to the required action (Drive or Stop, Right or Left). Another member of Applicants' research team was monitoring the recorded signal, which was transmitted wirelessly to their computer to ensure the quality. FIG. 2 illustrates the experimental procedure to collect the ECG data.

The total number of successfully completed recordings for each command was 440 recordings from all ten participants. The collected data was merged without separating them according to their corresponding participants. This way, Applicants examined the performance of the disclosed classification method in distinguishing between the four commands using a dataset from ten different subjects. For each command, the first 44 recordings were for S1, the second 44 recordings were for S2, the third 44 recordings were for S3, and so on, and the last 44 recordings were for S10. The recorded ECG dataset was split, labeled, stored, and prepared for the preprocessing stage.

ECG Preprocessing. An ECG Preprocessing stage is comprised of a combination of different noise attenuation and calibration approaches to prepare the ECG signals for further analysis. The raw ECG data are prone to noises and artifacts that arise due to instrumentation, electrode placement, power line, baseline wander, subject movement, or any other disturbance. Even though ECG acquisition devices are designed to reduce powerline interference, a very small amount of external interference is expected to affect the signal. The recorded ECG signals were analyzed using gHIsys MATLAB toolbox (https://www.gtec.at/product/ghisys/accessed on Jun. 1, 2023). For the above-mentioned ECG dataset, bipolar was applied between the left forehead and right forehead electrodes where voltage differences between the left forehead, right forehead, and left below neck were obtained. A 4th order Butterworth bandpass filter with 0.5 Hz to 150 Hz bandwidth was used to attenuate the baseline drift and the noisy signals from the ECG signals. Then, a notch filter at 60 Hz (the standard power frequency in Mississippi, USA) was used to minimize the effects of power frequency. The baseline wander is normally present with frequencies below 0.05 Hz and is generally caused by respiration or perspiration of the subject, or movement, which can be attenuated using a high pass filter. See X. Hu, Z. Xiao and N. Zhang, “Removal of baseline wander from ECG signal based on a statistical weighted moving average filter,” Journal of Zhejiang University—Science C, vol. 12, pp. 397-403, 2011.

The last preprocessing technique Applicants applied was removing the baseline wander from all the recorded datasets by applying a high-pass filter with a cut-off frequency of 0.5 Hz. Then, the filtered ECG datasets were segmented into a fixed 10 seconds (5000 samples) and stacked into an array. No further preprocessing operations were considered to avoid losing any features from the recorded ECG that can help increase the classification accuracy. The dataset was split into 1408 recordings for training and 352 recordings for testing (80% for training and 20% for testing).

Feature Extraction. Applicants performed feature extraction in the time domain to illuminate any risk of missing the dynamic changes in ECG due to the emotional status during inner speech activity. Multi-feature extraction methods were applied on one block for each recording with a time window of 10 s. Autoregressive model (AR) coefficients, Shannon Entropy (SE), fractal estimates, and multiscale wavelet variance estimates were used to extract features of the recorded data.

### AR Coefficients

In an AR method of order p, the signal X{n} at time n could be represented as a linear sequence of p prior estimates of the same signal. Specifically, the AR method is modeled as follows:

$$X \{ n \} = {\sum }_{j = 1}^{p} a \{ i \} x ( n - i ) + e \{ n \} \qquad ( 1 )$$

Where a{i} is i coefficients of the AR representation, e{n} is added noise with zero mean value, and p is the order number of the AR model. Many methods could be used to calculate the coefficients of an AR representation. The method Applicants used to estimate the AR order in this work is the ARfit. See A. Neumaier and T. Schneider, 2001: “Estimation of parameters and eigenmodes of multivariate autoregressive models,” ACM Trans. Math. Softw., vol. 27, no. 1, pp. 27-57, March 2001. The 4th order has been selected for the recorded EEG signals.

### Shannon Entropy

Entropy is estimated in the time domain, which generally breaks up the signal into multi-segments that are then compared for high similarity. Shannon entropy is one of the most attractive cost functions, which is a measure of signal complexity to wavelet coefficients generated by wavelet packet transform where larger entropy values represent higher process uncertainty and higher complexity. See D. Wang, D. Miao, and C. Xie, “Best basis-based wavelet packet entropy feature extraction and hierarchical EEG classification for epileptic detection,” Expert Systems with Applications, vol. 38, no. 11, pp. 14314-14320, 2011. The representation of the Shannon entropy for the undecimated wavelet packet transform is formulated as follows:

$${SE}_{j} = {\sum }_{k = 1}^{n} {P}_{jk} log {P}_{j k} \qquad ( 2 )$$

where n is the subsequent coefficients in a j number of nodes, and Pⱼₖ are the normalized squares of the wavelet packet coefficients in each node.

### Fractal Estimates

Fractal analysis is a powerful tool for the analysis of physiological signals since it gives a description of the singular behavior of a signal. A multifractal analysis can be used where the wavelet leaderbased multifractal formalism is estimated. See R. Leonarduzzi, G. Schlotthauer, and M. Torres, “Wavelet leader based multifractal analysis of heart rate variability during myocardial ischaemia,” Annu Int Conf IEEE Eng Med Biol Soc, vol. 2010, pp. 110-3, 2010. Using this method, the width of the singularity spectrum in the ECG was obtained from the discreet wavelet transform leader to estimate the multifractal nature of the signal, which can be estimated as follows:

$${L}_{X} ( j , k ) = {SUP}_{{∅}^{′} ∈ ∃ ∅} ‖ {d}_{x} ( j , k ) ‖ \qquad ( 3 )$$

Where L_{X}(j,k) is the wavelet leader, SUP_{Ø′∈∃Ø} are the time neighborhood parameters per sample (k+1), and dₓ is the wavelet coefficients. Then, Applicants used the 2nd order scale exponents to construct the power-law behavior in the signal at different resolutions. For the ECG with a length size of 10 s, two fractal measures obtained by wavelet methods were estimated for each recording.

Multiscale Wavelet Variance estimates (MWV)

Wavelet variance measures the variability in data by scale or, equivalently, variability in the ECG signal over octave-band frequency intervals. For a signal length of 10 s sampled at 0.5 KHz, 5000 samples were collected. The number 5000 lays between 2¹¹ and 2¹², considering 2¹² to extract the maximum possible number of features, and using the 4th order ‘db2’ wavelet (see I. Daubechies, “Ten Lectures on Wavelets,” SIAM, p. 194, 1992), 10 multiscale wavelet variance features were extracted from each recording using the following formula:

$$No . of Wavelet Variance = {2}^{A - db} \qquad ( 4 )$$

A total of 32 features were extracted from the ECG data per time window: 4 AR coefficients per time window, 16 SE values, 2 fractal estimates, and 10 wavelet variance estimations. After the multi-feature extracting stage, the ECG data was reconstructed to be a 1408-by-29 features matrix for training and a 352-by-29 features matrix for testing. By employing Autoregressive coefficients, Shannon Entropy, fractal estimates, and multiscale wavelet variance estimates, the data was reduced from 5000 to 29 element vectors. Representation of the class variance with the extracted features from the ECG data will be reported in the final results.

Classification. In the classification stage, the data was processed with supervised learning, where the support vector machine model was employed to learn from the prepared data. In this paper, the classification stage was defined as the determination of four different internally spoken commands (Drive, Stop, Right, and Left), which is a multi-class classification process. Applicants used a SVM, which is a machine learning algorithm for differentiation between the four chosen commands. SVM is one of the most well-known supervised learning algorithms specialized in classification problems. Classification using SVM is powered by generating a best line or decision boundary that is capable of segregating an n-dimension space to multi-classes to easily enable data sorting to the category to which they belong. See Y. Tan and J. Wang, “A support vector machine with a hybrid kernel and minimal vapnik-chervonenkis dimension,” IEEE Transactions on knowledge and data engineering, vol. 16, no. 4, pp. 385-395, 2004. SVM works on picking the margin points that construct vectors, which are called support vectors to assist with generating the best decision boundary.

The SVM algorithm applies a set of mathematical functions that are known as the kernel functions. The kernel function performs a kind of similarity measure between input objects and transforms it into the required output. See K. Grauman and T. Darrell, “The pyramid match kernel: Discriminative classification with sets of image features,” Tenth IEEE International Conference on Computer Vision (ICCV'05), vol. 1, no. 2, pp. 1458-1465, 2005. Applicants utilized a 10-fold cross-validation to evaluate the performance of the disclosed model successive to feature extraction of the preprocessed dataset. The k-fold cross-validation (k=10) was used to achieve an accurate estimate of the disclosed model performance on the recorded inner speech data and to avoid overfitting in the classification process.

Applicants implemented the disclosed classifier using MATLAB 2023a. With k-fold cross-validation of value 10, the model performance will be evaluated after randomly selecting 20% of the data (testing data) to 10 subsets while using the k−10 subsets for training the randomly selected 80% of the data (training data). This consideration can ensure that testing data will be entirely unknown to the classifier that is testing, and training data will not come from the same given group.

Performance Evaluation. Evaluation metrics adopted within a variety of deep learning techniques are critical in examining the reliability of the designed classifiers. To evaluate the trained model performance, estimating performance metrics was considered. See L. Alzubaidi et al., “Review of deep learning: Concepts, cnn architectures, challenges, applications, future directions,” Journal of Big Data, vol. 8, no. 1, pp. 1-74, 2021. The classified EEG data using the disclosed deep learning method was grouped into true positive (T_{P}), false positive (F_{P}), true negative (T_{N}), and false negative (F_{N}). The number of F_{P} and F_{N} are the samples that were misclassified, and the number of T_{P} and T_{N} are the samples that were correctly classified. The most state-of-art metrics for classification are accuracy, precision, recall, and F1-score. Accuracy estimates the percentage of correct predicted outputs to the overall number of samples in the processed dataset. Recall (sometimes called Sensitivity) estimates the percentage of T_{P} to the summation of T_{P} and F_{N}. Precision estimates the percentage of T_{P} to the summation of T_{P} and F_{P}. Hence, the F1-Score estimates the average between recall and precision.

Moreover, the Area Under Curve (AUC) of the Receiver Operating Characteristic (ROC) (AUC-ROC) was plotted. AUC-ROC is a common ranking type of metric that is utilized to show comparisons between learning algorithms and create an optimal learning model by exposing the entire classifier ranking performance. See V. Lawhern, W. Hairston, K. McDowell, W. Marissa, and K. Robbins, “Detection and classification of subject-generated artifacts in EEG signals using autoregressive models,” Journal of Neuroscience Methods, vol. 208, no. 2, pp. 181-189, 2012. Furthermore, while there is a multi-class classification task, areas under the curve were calculated and presented by macro-averaging, in which each corresponding metric for each individual class was estimated. The following formula is used to estimate the AUC-ROC value for multi-class problems:

$$ACU = {S}_{p} - {n}_{p} ( {n}_{n} - 1 ) / {Nn}_{p} {n}_{n} \qquad ( 5 )$$

Where Sₚ, nₚ, nₙ, and N represent the sum of all positive samples, positive samples, negative samples, and the number of classes, respectively.

### Results

Next, Applicants further investigated the generalization and consistency of the performance of the designed model across different subject datasets. The disclosed model for ECG-based inner speech recognition signals, including the multi-features representation of the preprocessed ECG signal and the SVM for the classification and determination of different inner speech commands, is disclosed herein.

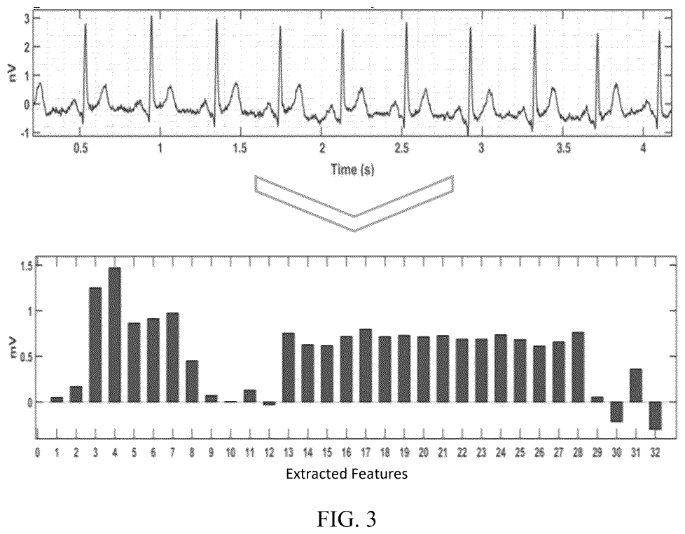

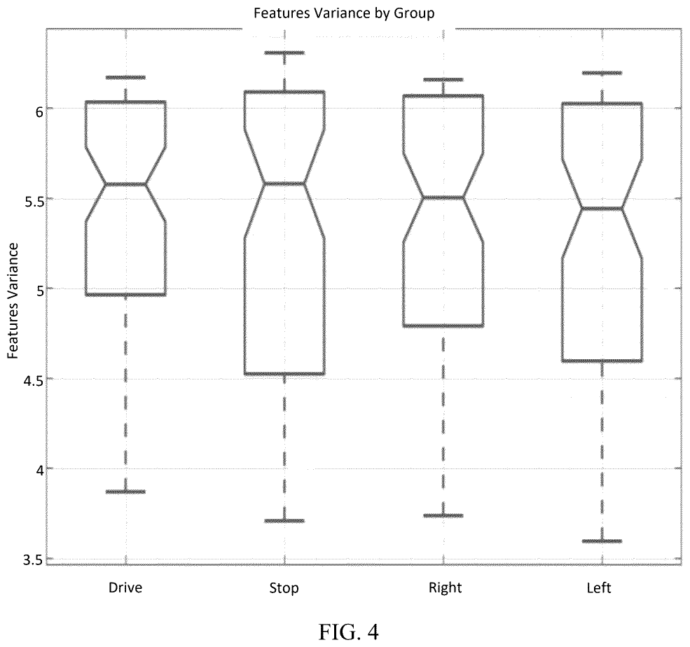

Feature Extraction results. FIG. 3 shows 4 s samples (for S1, the first session of performing the Drive command) and the 32 features representation obtained from each recording of it using the disclosed multi-feature extraction methods. The first four features are AR coefficients, features 5 to 20 are Shannon Entropy, 21 and 22 are fractal estimates, and the last 10 features represent the multiscale wavelet variance. Every feature vector of the same class should be closer in its representation point, and they should be far from each other in different classes. For precisely monitoring the variance in data distribution of all features in the four classes, a boxplot was used. FIG. 4 is a boxplot for a sample of the variance between each individual command using the extracted features. The obtained results using the disclosed features extraction methods showed a noticeable variation between the four commands, which accordingly will assist with distinguishing between them and improve the classification accuracy.

Classification results. Applicants fit a multi-class quadratic SVM to only 80% of the data and then used that model to make predictions on the remaining 20% of the data. The SVM was utilized to use a polynomial kernel function, C=2 and gamma=0.1, which was selected as the estimator. Both gamma and C are regularization parameters where gamma determines the width of the kernel function, and C controls the trade-off between achieving a simple decision boundary and a good fit to the data during the training process.

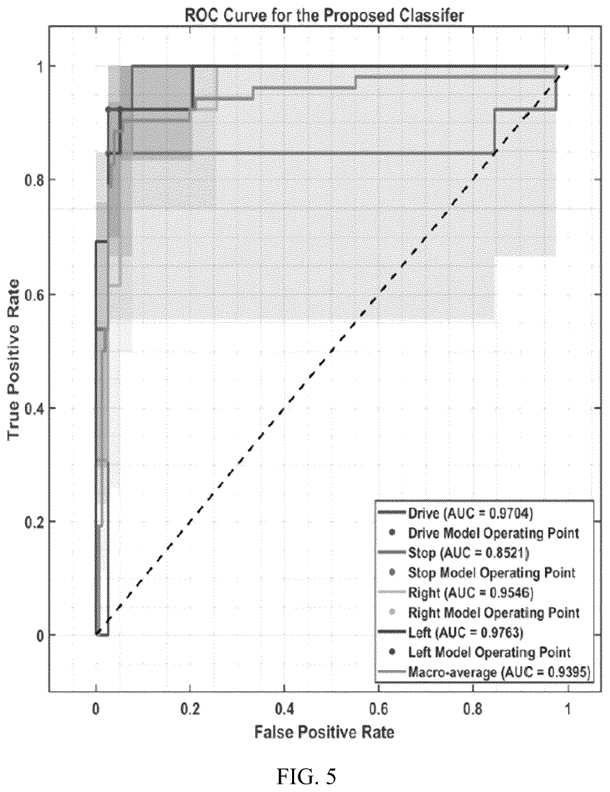

The highest performance of the model was achieved by feeding the features selected by Autoregressive coefficients, Shannon Entropy, fractal estimates, and multiscale wavelet variance with an overall accuracy of 88.46%, an overall precision of 88.64%, overall recall of 88.58%, and overall F1-score of 88.52%. Table 1 shows the precision, recall, and F1-score for each individual class that Applicants obtained with the disclosed feature-extracting methods. In addition, the macroaverage ROC-AUC of the model was 93.95%, which represents the macro-average of the ten folds. The model showed significantly an excellent performance using the extracted features by the disclosed feature extraction methods. The ROC-ACU plot illustrates the performance of the disclosed classifier in FIG. 5.

TABLE I
THE MODEL PERFORMANCE OF THE SVM MODEL WITH
THE DISCLOSED FEATURES EXTRACTION METHOD

| | Precision | Recall | F1 Score |
|---|---|---|---|
| DRIVE | 93.443 | 86.364 | 89.764 |
| STOP | 67.123 | 74.242 | 70.504 |
| LEFT | 67.164 | 68.182 | 67.669 |
| RIGHT | 90.476 | 86.364 | 88.372 |

Now, therefore, the following is claimed:

1. An internalized speech recognition method using a unimodal signal comprising the steps of:
placing at least one electrode on an individual;
collecting ECG data from the individual using the at least one electrode;
extracting features from the collected ECG data using at least one feature extraction method; and
classifying the features using supervised learning using a machine learning algorithm.

2. The method of claim 1, wherein the method further comprises the step of preprocessing the collected ECG data prior to extracting features, wherein the preprocessing comprises noise attenuation and calibration.

3. The method of claim 2, wherein the noise attenuation and calibration comprises the steps of applying a 4th order Butterworth bandpass filter with 0.5 Hz to 150 Hz bandwidth, applying a notch filter at 60 Hz, and applying a high-pass filter with a cut-off frequency of 0.5 Hz.

4. The method of claim 1, wherein the method further comprises presenting an audio or visual prompt to the individual prior to collecting ECG data.

5. The method of claim 1, wherein the at least one electrode comprises three electrodes.

6. The method of claim 5, wherein the electrodes are placed on the left side of the individual's forehead, the right side of the individual's forehead, and on the individual's left side below the neck.

7. The method of claim 1, wherein the at least one feature extraction method is selected from the group consisting of: autoregressive coefficient (AR), Shannon entropy, fractal measures, and multiscale wavelet variance estimation.

8. The method of claim 1, wherein the at least one feature extraction method comprises autoregressive coefficient (AR), Shannon entropy, fractal measures, and multiscale wavelet variance estimation.

9. The method of claim 1, wherein the machine learning algorithm is SVM.

10. The method of claim 1, wherein the individual has been diagnosed with or suspected of having a speech disorder.

11. The method of claim 1, wherein the individual has a disability that prevents or inhibits coherent speech.

12. The method of claim 1, wherein the features are classified using a predetermined set of words.

13. An internalized speech recognition method using a unimodal signal comprising the steps of:
placing at least one electrode on an individual;
collecting ECG data from the individual using the at least one electrode;
preprocessing the collected ECG data, wherein the preprocessing comprises noise attenuation and calibration;
extracting features from the collected ECG data using at least one feature extraction method, wherein the at least one feature extraction method is selected from the group consisting of: autoregressive coefficient (AR), Shannon entropy, fractal measures, and multiscale wavelet variance estimation; and
classifying the features using supervised learning using a machine learning algorithm, wherein the machine learning algorithm is SVM.

14. The method of claim 13, wherein the noise attenuation and calibration comprises the steps of applying a 4th order Butterworth bandpass filter with 0.5 Hz to 150 Hz bandwidth, applying a notch filter at 60 Hz, and applying a high-pass filter with a cut-off frequency of 0.5 Hz.

15. The method of claim 13, wherein the method further comprises presenting an audio or visual prompt to the individual prior to collecting ECG data.

16. The method of claim 13, wherein the at least one electrode comprises three electrodes.

17. The method of claim 16, wherein the electrodes are placed on the left side of the individual's forehead, the right side of the individual's forehead, and on the individual's left side below the neck.

18. The method of claim 13, wherein the individual has been diagnosed with or suspected of having a speech disorder.

19. The method of claim 13, wherein the individual has a disability that prevents or inhibits coherent speech.

20. The method of claim 13, wherein the features are classified using a predetermined set of words.
